SEQUENCE LISTING (1 sequence)

SEQ ID NO: 1 - DNA, 1448 bp, Limosilactobacillus reuteri
  source 1..1448: mol_type = genomic DNA
aatgggccaaggtcggtacggcactggcccaactagattgatggtgcttgcacctgattgacgatggatcaccagtgagtggcggacgggtgagtaacacgtaggtaacctgccccggagcgggggataacatttggaaacagatgctaataccgcataacaacaaaagccgcatggcttttgtttgaaagatggctttggctatcactctgggatggacctgcggtgcattagctagttggtaaggtaacggcttaccaaggcgatgatgcatagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttggagaagaacgtgcgtgagagtaactgttcacgcagtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgctaaccttagagataaggcgttcccttcggggacgcaatgacaggtggtgcatggtcgtcgtcagctcgtgtcgtgagatgttgggttaagtcccgcaacgagcgcaacccttgttactagttgccagcattaagttgggcactctagtgagactgccggtgacaaaccggaggaaggtggggacgacgtcagatcatcatgccccttatgacctgggctacacacgtgctacaatggacggtacaacgagtcgcaaactcgcgagagtaagctaatctcttaaagccgttctcagttcggactgtaggctgcaactcgcctacacgaagtcggaatcgctagtaatcgcggatcagcatgccgcggtgaatacgttcccgggccttgtacacaccgcccgtcacaccatgggagtttgtaacgcccaaagtcggtggcctaacctttatggtaggc

(19) United States
(12) Patent Application Publication
(10) Pub. No.: US 2025/0222044 A1
(43) Pub. Date: Jul. 10, 2025

(54) LIMOSILACTOBACILLUS REUTERI GOLDGUT-LR99 AND APPLICATION THEREOF

(71) Applicant: SHENZHEN PORSHEALTH BIOENGINEERING CO., LTD., SHENZHEN, GUANGDONG (CN)

(72) Inventors: GUOXUN XIAO, SHENZHEN, GUANGDONG (CN); YANYI ZHENG, SHENZHEN, GUANGDONG (CN); SILU ZHANG, SHENZHEN, GUANGDONG (CN); XIAOLI ZHOU, SHENZHEN, GUANGDONG (CN); ZHIZHU ZHANG, SHENZHEN, GUANGDONG (CN)

(21) Appl. No.: 19/008,966

(22) Filed: Jan. 3, 2025

(30) Foreign Application Priority Data
Jan. 5, 2024 (CN) 202410029515.8

(51) Int. Cl. A61K 35/744; C12N 1/20; C12R 1/01

(52) U.S. Cl. CPC A61K 35/744; C12N 1/205; C12R 2001/01

(57) ABSTRACT

The disclosure belongs to the technical field of biological medicine, and provides a strain of Limosilactobacillus reuteri GOLDGUT-LR99 which was deposited in China General Microbiological Culture Collection Center (CGMCC) on Dec. 18, 2023, wherein the deposit number is CGMCC No. 29346, and the deposit address is No. 3, Courtyard 1, West Beichen Road, Chaoyang District, Beijing. The strain of the disclosure has the functions of treating chronic fatigue syndrome, irritable bowel syndrome and the like.

### SEQUENCE LISTING

The sequence listing is submitted as a XML file filed via EFS-Web, with a file name of “Sequence-Listing.XML”, a creation date of Jan. 3, 2025, and a size of 3,590 bytes. The sequence Listing filed via EFS-Web is a part of the specification and is incorporated in its entirety by reference herein.

### TECHNICAL FIELD

The present disclosure belongs to the technical field of biological medicine.

### BACKGROUND ART

Chronic fatigue syndrome (CFS) is a complex, multi-system debilitating disease that is easily overlooked, primarily manifested by severe fatigue, cognitive dysfunction, gastrointestinal disorders. Heretofore, the diagnostic criteria and pathogenesis of CFS have not been known, and there are no targeted specific drugs [1-2].

Patients suffering from chronic fatigue syndrome often have irritable bowel syndrome (IBS), a clinically common chronic bowel dysfunction disease with symptoms of abdominal pain, abdominal discomfort and altered bowel function, which greatly affects people's life [3-5].

The existing treatment strategies are often limited to the treatment of corresponding symptoms (anti-fatigue, analgesic, anxiolytic drugs), have limited efficacy and certain side effects, and furthermore, due to the heterogeneity of patients, it is difficult to have effective treatment options for all patients.

### SUMMARY

In order to effectively improve or treat chronic fatigue syndrome and irritable bowel syndrome, the first object of the present disclosure is to provide Limosilactobacillus reuteri GOLDGUT-LR99 which has been deposited in China General Microbiological Culture Collection Center on Dec. 18, 2023, the center is abbreviated as CGMCC, the deposit number is CGMCC No. 29346, and the deposit address is No. 3, Courtyard 1, West Beichen Road, Chaoyang District, Beijing.

In a specific embodiment of the disclosure, the disclosure also provides the application of the Limosilactobacillus reuteri GOLDGUT-LR99 in preparing health food or medicine for treating or improving chronic fatigue syndrome.

In a specific embodiment of the present disclosure, further, the chronic fatigue syndrome includes symptoms of inhibited body weight gain, symptoms of impaired spatial memory, and symptoms of impaired exercise property.

In a specific embodiment of the disclosure, the third aspect of the disclosure provides the application of the Limosilactobacillus reuteri GOLDGUT-LR99 in preparing health food or medicine for treating or improving irritable bowel syndrome.

In a specific embodiment of the present disclosure, further, the irritable bowel syndrome includes symptoms of inhibited body weight gain, symptoms of visceral hypersensitivity, symptoms of anxiety, and symptoms of intestinal barrier dysfunction.

In a specific embodiment of the disclosure, the fourth aspect of the disclosure provides the application of the Limosilactobacillus reuteri GOLDGUT-LR99 in preparing a bacterial agent for inhibiting Escherichia coli or Pseudomonas aeruginosa.

The Limosilactobacillus reuteri GOLDGUT-LR99 disclosed by the disclosure can improve the memory and cognitive functions of CFS hosts and improve the exercise coordination property of the hosts, and can also improve visceral hypersensitivity and anxiety state of IBS host, and improve intestinal barrier function of host.

### BRIEF DESCRIPTION OF THE DRAWINGS

FIG. 1 shows colony morphology of the Limosilactobacillus reuteri GOLDGUT-LR99 strain.

FIG. 2 shows an exemplary graph of colon tissue immunohistochemistry results in the experiment of Limosilactobacillus reuteri GOLDGUT-LR99 strain treating IBS mice.

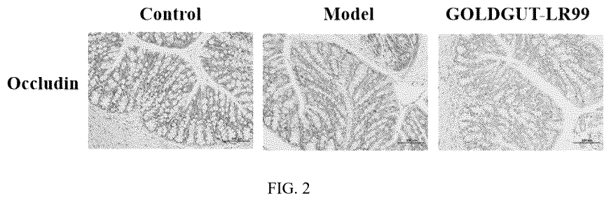

### DETAILED DESCRIPTION OF THE EMBODIMENTS

The specific embodiment is as follows:

Modified MRS liquid medium (g/L): 52.4 g of MRS broth, 0.01 g of hemin, 0.001 g of resazurin, 10% (v/v) of clarified rumen fluid, added with distilled water to 1 L, pH 7.2±0.1, sterilized at 115° C. for 25 min, and then used after autoclaving. An additional 15 g of agar was added to obtain solid medium.

### 1. Acquisition, Isolation and Identification of Strain

The strain was obtained by anaerobic separation of fresh feces provided by healthy feces donors (who signed the informed consent form themselves) from Qingdao in Shandong on Jan. 17, 2022, by the patent inventor.

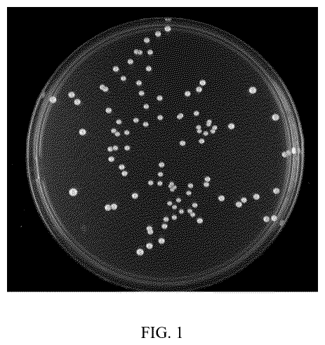

Colony morphology: in modified MRS broth medium (each liter of the modified MRS broth medium: 52.4 g of MRS broth, 0.01 g of hemin, 0.001 g of resazurin, 10% (v/v) of clarified rumen fluid, added with distilled water to 1 L, pH 7.2±0.1, sterilized at 115° C. for 25 min, and then used after autoclaving. An additional 15 g of agar was added to obtain solid medium), culturing was carried out at 37° C. for 2-3 days under anaerobic conditions, the diameter of the colony was 2-3 mm, the colony was white, smooth, and had no water-soluble pigment (FIG. 1).

Molecular identification method: the full sequence analysis of 16S rRNA was conducted, the identification classification was named Limosilactobacillus reuteri, and the strain was named GOLDGUT-LR99. This strain has been deposited in China General Microbiological Culture Collection Center (hereinafter referred to as: CGMCC) on Dec. 18, 2023, with the deposit number of CGMCC No. 29346, and the deposit address of No. 3, Courtyard 1, West Beichen Road, Chaoyang District, Beijing. Namely, the patent strain deposit number is: CGMCC No. 29346; the 16S rRNA sequence homology with the standard strain of Limosilactobacillus reuteri is 99%, and the sequence is shown in SEQ ID No. 1 in the sequence list.

### 2. Culture and Preservation Method

Limosilactobacillus reuteri was activated twice in the modified MRS broth, then inoculated into the modified MRS liquid medium with the same composition at an inoculum size of 0.1%-10%, and anaerobically incubated at 37° C. for 2 days.

Method for long-term storage of the bacteria: after the bacterial solution was centrifuged at 10,000 rpm at 4° C. for 2 minutes to collect the precipitated cells, the precipitated cells were washed with a PBS buffer solution, resuspended in a solution containing 15-25% skim milk powder at a ratio of 5:1, freeze-dried and stored in a sealed manner, or frozen with liquid nitrogen in a solution containing 15% glycerol and 85% serum and stored at −80° C.

### 3. Application and Effect of the Strain:

3.1 Screening of Limosilactobacillus reuteri GOLDGUT-LR99 Strain and Animal Experiment of Limosilactobacillus reuteri GOLDGUT-LR99 Alleviating Chronic Fatigue Syndrome

### 3.1.1 Materials and Methods:

1) Modified MRS Liquid Medium (g/L):

52.4 g of MRS broth, 0.01 g of hemin, 0.001 g of resazurin, 10% (v/v) of clarified rumen fluid, added with distilled water to 1 L, pH 7.2±0.1, sterilized at 115° C. for 25 min, and then used after autoclaving. An additional 15 g of agar was added to obtain solid medium

2) Preparation Method of Limosilactobacillus reuteri GOLDGUT-LR99 Bacterial Powder Feed

Limosilactobacillus reuteri was activated twice in the modified MRS broth medium, then inoculated into the modified MRS liquid medium with the same composition at an inoculum size of 0.1%-10%, and anaerobically incubated at 37° C. for 2 days. After the bacterial solution was centrifuged at 10,000 rpm at 4° C. for 2 minutes to collect the precipitated cells, the precipitated cells were washed with a PBS buffer solution, resuspended in a solution containing 15-25% skim milk powder at a ratio of 5:1 and freeze-dried, then the number of viable bacteria was tested, and the ontained bacterial powder was mixed into the feed.

### 3) Acid-Base Resistance Test

PBS (Na₂HPO₄ 8 mM, NaCl 136 mM, KH₂PO₄ 2 mM, KCl 2.6 mM) with anaerobic pH of 2, 2.5, 3, 3.5, 4, 7, 9 and 10 was prepared, with a total of eight gradients and the group of pH=7 as a control; each seed solution was subpackaged into 1.5 mL EP tube at a ratio of 0.1 mL/tube, centrifuged at 6000 rpm for 5 min to remove supernatant, with 3 parallel item for each seed solution; the cells were resuspended using 1 mL of PBS with different pH and incubated for 4 h at 37° C.; the bacterial suspension was subjected to gradient dilution by using PBS with pH=7 so as to balance the pH value of the bacterial suspension; 50 μL of bacterial suspension was coated in MRS solid culture medium; after the incubation at 37° C. for 24-48 h and the formation of visible colony, counting was performed to calculate survival rate.

### 4) Carbon Source Utilization and Oxidation Experiment

The AN Biolog micro-pore plate was used for measuring the utilization and oxidation conditions of different carbon sources of Limosilactobacillus reuteri GOLDGUT-LR99, the AN microplate used nutrients and biochemical reagents, and the method included the following steps: 1 mL of Limosilactobacillus reuteri GOLDGUT-LR99 bacterial liquid of the strain to be detected at the end of logarithmic growth was pipetted and centrifuged, then the supernatant was removed, the cells was washed three times with the seed liquid and suspended with 1 mL of the seed liquid, 50 μL of the suspension was taken and diluted in 10 mL of the seed liquid, the diluent was subpacked in a Biolog plate (100 μL/pore) after mixing uniformly, and the Biolog plate with the diluent was placed in 37° C. for anaerobic culture for 24 hours. After the culture was finished, OD₅₉₀ and OD₇₅₀ were detected by using microplate reader, the numerical values were recorded, and the utilization conditions of carbohydrate, organic acid and other substances of the Limosilactobacillus reuteri GOLDGUT-LR99 were calculated, wherein the calculation method was as follows: Y=OD₅₉₀−OD₇₅₀;

### 5) Antibacterial Property Test

Limosilactobacillus reuteri GOLDGUT-LR99 was inoculated at 1% (v/v) into anaerobic glass tubes of modified MRS liquid medium and incubated at 37° C. for 12 h. Pathogenic strains such as Escherichia coli and Pseudomonas aeruginosa were respectively inoculated in a liquid LB medium, cultured on a constant temperature shaker overnight at 37° C. with a rotational speed of 250 rpm, and then the bacterial suspension was prepared. MRS solid medium was cooled to about 55° C., mixed with pathogenic bacteria suspension at a certain ratio to make the number of viable bacteria of the system be 106 CFU/mL, then rapidly poured into a flat plate in which an oxford cup was placed in advance, the oxford cup was taken out after the culture medium was cooled and solidified, and 200 μL of Limosilactobacillus reuteri GOLDGUT-LR99 fermentation liquor was injected into each hole and covered lightly by the flat plate, then the same was placed in a constant temperature incubator at 37° C. and observed after culturing for 24 hours, and the diameter of a bacteriostasis ring was measured by using a vernier caliper, wherein the data were shown in table 4.

### 6) Metabolite Detection

Firstly, ethyl acetate was added in a 1:1 equal volume for extraction; vortex oscillation was performed for 15 min each time, standing was performed for 2 min at intervals; centrifuging was performed at 10000 rpm for 10 min; the supernatant was pipetted and passed through an organic filter membrane; the GC/MS analysis was performed with 300-400 μL of the organic phase.

### 7) Antibiotic Sensitivity

Antibiotic sensitivity of Limosilactobacillus reuteri GOLDGUT-LR99 was determined using the K-B method (Kirby-Bauer method).

### 8) Design of Animal Experiment

SPF-grade wild female C57BL/6J mice were adopted for the experiment, 4-6 weeks, and the mice were adaptively fed for one week;

Animal adaptation: before the experimental formally began, the model mice (except for control group) were placed into the interference box of the sleep depriver for adaptation for three consecutive days, 3 hours per day.

Modeling and intervention: from the first week to the end of the experiment, the GOLDGUT-LR99 group was given mixed quantitative bacterial powder feed (viable count 2×10⁹ CFU/day/PCS); mice in the control group and model group were given a sterile powder feed daily.

From 2 to 10 weeks after the start of the experiment, all mice in the model group and GOLDGUT-LR99 group were subjected to sleep deprivation, with 23 hours of sleep deprivation and light interference per day, and 1 hour of rest.

### 9) Indicator Detection

Behavioral detection: elevated plus maze testing; Y-maze spontaneous alternation experiment; Y-maze new different arm identification experiment; rotarod experiment.

Y-maze spontaneous alternation experiment: mice were evaluated for memory and cognitive property. The mice were placed from the end of one arm and were freely explored for 8 min, with the experimenters out of the sight of the mice. The track of action was tracked by camera shooting, and the sequence number of each arm entered by the mouse was recorded in turn. Thus, the total number of times of entering the channel, the total movement distance and the spontaneous alternation rate were counted. Spontaneous alternation rate (%)=total times of three-strand conversion with all three arm serial numbers/possible times of conversions entering into maze arms (total number of entering arms−2)×100%

Y-maze new different arm identification experiment: mice were evaluated for memory property using the natures of animals exploring new areas. The experiment was divided into two parts, namely, the first stage of the experiment was a training period and the second stage was a detection period. New different arm: during the first stage of the experiment, namely the training period, it was covered by a baffle plate, and during the second stage, namely the detection period, it was opened; starting arm: the arm where the mouse was located when entering the maze. The starting arm and other arms were always open throughout the experiment, allowing the animal to enter and exit freely. A camera lens was arranged at a position 1.5 m above the maze to take video during the entire process. Training period: the new different arm was blocked by the baffle plate, the mice were placed from the starting arm and move freely in the starting arm and other arms for 10 minutes; after the training, the mice were placed back into the cage. The second stage, namely, the testing period, was carried out 20 hours later. Detection period: the baffle of the new different arm was removed, the mice were placed from the starting arm and freely explored in the three arms for 5 minutes. The movement distance of each mouse in each arm within 5 min was recorded on video, and the movement distance in the new different arm was used as an evaluation indicator.

Rotarod experiment: mice were evaluated for their property to coordinate exercise and extent of fatigue. Each mouse was placed on a rotarod fatigue machine at a rotation speed of 30 rpm for a fatigue rotarod training of the mouse once per day for three consecutive days. Then the formal rotarod experiment was performed, the mice were placed on an electric rotarod fatigue instrument with the rotating speed of 1 rpm increased to 30 rpm, the time from the beginning of the experiment to the falling of the mice was recorded as rotarod stay time, and the stay time was taken as an evaluation indicator for the states of the mice.

### 3.1.2 Experimental Results:

1) Limosilactobacillus reuteri GOLDGUT-LR99 Acid Resistance Property:

the data in Table 1 shows that Limosilactobacillus reuteri GOLDGUT-LR99 had excellent acid resistance, and the survival rate could reach over 93% after being incubated in acid solution with pH value of 3.0-4.0 for 3 h.

TABLE 1
Acid resistance of Limosilactobacillus reuteri GOLDGUT-LR99

| | Survival rate 1 | Survival rate 2 | Survival rate 3 | Average survival |
|---|---|---|---|---|
| pH | (%) | (%) | (%) | rate (%) |
| 3 | 94.55 | 93.37 | 93.96 | 93.96 ± 0.48 |
| 3.5 | 97.13 | 96.57 | 95.19 | 96.3 ± 0.82 |
| 4 | 99.28 | 98.29 | 99.38 | 98.98 ± 0.49 |

2) Base Resistance of Limosilactobacillus reuteri GOLDGUT-LR99

The data in Table 2 shows that Limosilactobacillus reuteri GOLDGUT-LR99 had excellent base resistance, and the survival rate could reach over 97% after being incubated in base solution with pH value of 9.0-10.0 for 4 h.

TABLE 2
Base resistance of Limosilactobacillus reuteri GOLDGUT-LR99

| | Survival rate 1 | Survival rate 2 | Survival rate 3 | Average survival |
|---|---|---|---|---|
| pH | (%) | (%) | (%) | rate (%) |
| 9 | 99.69 | 99.65 | 99.73 | 99.69 ± 0.03 |
| 10 | 97.57 | 96.41 | 99.17 | 97.72 ± 1.13 |

3) Metabolic Fingerprint of Limosilactobacillus reuteri GOLDGUT-LR99

The data result in the Table 3 shows that Limosilactobacillus reuteri GOLDGUT-LR 99 had better utilization effect on D-fructose, turanose, L-trehalose and the like; could effectively utilize D,L-lactic acid, D-glucaric acid and the like; had good utilization effect on L-valine in amino acids.

TABLE 3
Metabolic fingerprint of Limosilactobacillus reuteri GOLDGUT-LR99

| Substrate | Utilization | Substrate | Utilization | Substrate | Utilization | Substrate | Utilization |
|---|---|---|---|---|---|---|---|
| Water | − | Hexanediol | − | Glycerol | − | α-Methyl-D- | − |
| | | | | | | galactoside | |
| N-Acetyl-D- | − | i-Erythritol | − | D,L-α- | − | β-Methyl-D- | − |
| galactosamine | | | | Glycerophosphate | | galactoside | |
| N-Acetyl-D- | − | D-Fructose | ++ | m-Inositol | + | α-Methyl-D- | − |
| glucosamine | | | | | | glucoside | |
| N-Acetyl-β-D- | − | L-Trehalose | ++ | α-D-Lactose | − | β-Methyl-D- | − |
| mannosamine | | | | | | glucoside | |
| Ribitol | − | D-Galactose | ++ | Lactulose | − | Palatinose | ++ |
| Amygdalin | − | D-Galacturonic | ++ | Maltose | − | D-Melibiose | − |
| | | acid | | | | | |
| D-Arabinitol | − | Ergosterol | ++ | Maltotriose | ++ | L-Rhamnose | ++ |
| Arbutin | − | D-Glucoronate | ++ | D-Mannitol | + | Salicin | − |
| D-Cellobiose | ++ | D-Aminoglucuronate | ++ | D-Gluconolactone | ++ | D-Sorbitol | ++ |
| α-Cyclodextrin | ++ | α-D-Glucose | − | D-Melezitose | − | Stachyose | + |
| β-Cyclodextrin | − | Glucose-1- | + | D-Melibiose | ++ | Sucrose | − |
| | | phosphate | | | | | |
| Dextrin | − | Glucose-6- | ++ | 3-Methyl-D- | ++ | D-Trehalose | − |
| | | phosphate | | 4-glucose | | | |
| Melezitose | ++ | Methyl lactate | + | L-Alanineamide | ++ | L-Methionine | − |
| Ethanoic acid | − | D-Malic acid | − | L-Alanine | − | L-Phenylalanine | − |
| Formic acid | − | L-Malic acid | − | L-Alanyl-L- | − | L-Serine | − |
| | | | | M-glutamate | | | |
| Fumaric acid | − | Propionic acid | − | L-Alanyl-histidine | − | L-Threonine | − |
| Glyoxylic acid | + | Pyruvic acid | + | L-Alanyl-L- | − | L-Valine | + |
| | | | | M-threonine | | | |
| α-Hydroxybutyric | − | Methyl acetate | ++ | L-Asparagine | + | L-Valine + L- | − |
| acid | | | | acid | | M-Aspartamid acid | |
| β-Hydroxybutyric | − | D-Glucarate | ++ | L-Glutamic acid | − | 2′- | − |
| acid | | | | | | Deoxyadenosine | |
| Itaconic acid | − | Succinamide | + | L-Glutamate salt | − | Inosine | − |
| | | acid | | | | | |
| α-Ketoglutaric | − | α-Succinic | − | Glycine-L-aspartic | − | Thymidine | − |
| acid | | acid | | acid | | | |
| α-Ketovaleric acid | − | Succinic acid | − | Glycyl-L-glutamine | − | Uridine | − |
| | | methyl ester | | | | | |
| D,L-Lactic acid | ++ | m-Tartaric acid | + | Glycyl-L-methionine | − | Uridine-5′- | − |
| | | | | | | monophosphate | |
| L-Lactic acid | ++ | L-Uracilic acid | − | Glycylprolline | − | Uridine-5′- | − |
| | | | | | | monophosphate | |

Notes:
when Y > 0.1, it is shown that the strain was able to effectively utilize this substance, marked as++; when 0.1 > Y > 0.05, it is shown that the strain was able to better utilize this substance, marked as +; when Y < 0.05, it is shown that the strain was not able to utilize this substance, marked as −.

4) Limosilactobacillus reuteri GOLDGUT-LR99 antibacterial property:

The data in Table 4 shows that Limosilactobacillus reuteri GOLDGUT-LR99 could effectively inhibit common opportunistic pathogens.

TABLE 4
Antibacterial Property of Limosilactobacillus reuteri
GOLDGUT-LR99 (cm)

| Pseudomonas aeruginosa | Escherichia coli |
|---|---|
| 1.0 | 0.9 |

5) Metabolite Detection of Limosilactobacillus reuteri GOLDGUT-LR 99:

The data in Table 5 shows that Limosilactobacillus reuteri GOLDGUT-LR99 had property for producing high amounts of acetic acid and lactic acid and for producing propionic acid, isobutyric acid, butyric acid and isovaleric acid.

TABLE 5
Acid production property of Limosilactobacillus reuteri GOLDGUT-LR99 (mg/L)

| Acetic | Propionic | Isobutyric | Butyric | Isovaleric | Lactic | Succinic |
|---|---|---|---|---|---|---|
| acid | acid | acid | acid | acid | acid | acid |
| 528.44 | 0.8 | 0.4 | 1 | 0.53 | 1028.2 | 6.2 |

6) Antibiotic Resistance of Limosilactobacillus reuteri GOLDGUT-LR99:

The data in Table 6 shows that Limosilactobacillus reuteri GOLDGUT-LR99 was sensitive to a variety of antibiotics including ampicillin, tetracycline, etc.

TABLE 6
Antibiotic susceptibility test results

| Antibiotic | Result | Antibiotic | Result | Antibiotic | Result |
|---|---|---|---|---|---|
| Penicillin | S | Ceftriaxone | S | Midecamycin | R |
| Benzylpenicillin | S | Cefperazone | S | Norfloxacin | R |
| Ampicillin | S | Butikam | S | Ofloxacin | R |
| Carbenicillin | S | Gentamicin | S | Ciprofloxacin | R |
| Piperacillin | S | Kanamycin | S | Vancomycin | R |
| Cephalexin | S | Neomycin | S | Polymyxin | R |
| Cefazolin | S | Tetracycline | S | Compound | R |
| | | | | Sulfamethoxazole | |
| Cefradine | S | Doxycycline | S | Furazolidone | S |
| Cefuroxime | S | Minocycline | S | Chloramphenicol | S |
| Ceftazidime | S | Erythromycin | R | Clindamycin | R |

Notes:
R represents resistance and S represents sensitivity.

7) Improvement on Slow Weight Gain in Mice with Chronic Fatigue Syndrome by Limosilactobacillus reuteri GOLDGUT-LR99

The results in Table 7 show that Limosilactobacillus reuteri GOLDGUT-LR99 could significantly increase the weight of mice, and the weight growth of mice in the model group was significantly inhibited.

TABLE 7
Weights (g) of mice in each group

| Mouse ID | No. 1 | No. 2 | No. 3 | No. 4 | No. 5 | Average | P-value | |
|---|---|---|---|---|---|---|---|---|
| Control group | 20.98 | 20.56 | 19.21 | 19.88 | 19.33 | 19.99 ± 0.69 | <0.0001 | |
| Model group | 16.68 | 16.71 | 16.6 | 16 | 16.51 | 16.5 ± 0.26 | | <0.0001 |
| GOLDGUT-LR99 | 18.72 | 19.75 | 19.15 | 19.95 | 20.28 | 19.57 ± 0.56 | | |
| group | | | | | | | | |

8) Improvement on Memory and Cognitive Function in Mice with Chronic Fatigue Syndrome by Limosilactobacillus reuteri GOLDGUT-LR99:

The data in Tables 8 and 9 show that the spontaneous alternation rate in the Y-maze experiment of the mice in Limosilactobacillus reuteri GOLDGUT-LR99 group could be significantly improved; and the movement distance in the new different arm identification experiment could be obviously improved, which shows that the Limosilactobacillus reuteri GOLDGUT-LR99 could improve the condition of the spatial memory damage of the mice with chronic fatigue syndrome.

TABLE 8
Spontaneous alternation rate of model mice

| Mouse ID | No. 1 | No. 2 | No. 3 | No. 4 | No. 5 | Average | P-value | |
|---|---|---|---|---|---|---|---|---|
| Control group | 62.16 | 51.85 | 56.52 | 55.17 | 60 | 57.14 ± 3.62 | <0.0001 | |
| Model group | 37.14 | 37.5 | 27.27 | 33.33 | 38.5 | 34.75 ± 4.13 | | <0.0001 |
| GOLDGUT-LR99 | 51.61 | 54.17 | 50 | 55.56 | 48.15 | 51.90 ± 2.69 | | |
| group | | | | | | | | |

TABLE 9
Movement distance in new different arm of model mice

| Mouse ID | No. 1 | No. 2 | No. 3 | No. 4 | No. 5 | Average | P-value | |
|---|---|---|---|---|---|---|---|---|
| Control group | 548.87 | 663.26 | 554.47 | 778.28 | 558.69 | 620.71 ± 89.48 | <0.0001 | |
| Model group | 437.48 | 260.08 | 284.2 | 286.31 | 310.1 | 315.63 ± 62.95 | | <0.0001 |
| GOLDGUT-LR99 | 638.25 | 619.68 | 577.03 | 730.74 | 642.96 | 641.73 ± 50.22 | | |
| group | | | | | | | | |

9) Improvement on Exercise Ability of Mice with Chronic Fatigue Syndrome by Limosilactobacillus reuteri GOLDGUT-LR99:

The results in Table 10 show that the Limosilactobacillus reuteri GOLDGUT-LR99 group could significantly prolong the rotarod stay time of the mice in the rotarod experiment, and show that the mice had the effect of relieving fatigue symptoms of CFS mice.

TABLE 10
Stay time of model mice

| Mouse ID | No. 1 | No. 2 | No. 3 | No. 4 | No. 5 | Average | P-value | |
|---|---|---|---|---|---|---|---|---|
| Control group | 420 | 420 | 402.19 | 420 | 420 | 416.44 ± 7.12 | <0.0001 | |
| Model group | 81.04 | 118.06 | 136 | 126.84 | 49.48 | 102.28 ± 32.34 | | 0.0002 |
| GOLDGUT-LR99 | 335.42 | 235.02 | 339.40 | 178.16 | 248.02 | 267.20 ± 61.97 | | |
| group | | | | | | | | |

3.2 Animal Experiment of Limosilactobacillus reuteri GOLDGUT-LR99 Alleviating Irritable Bowel Syndrome

### 3.2.1 Materials and Methods:

1) Modified MRS Liquid Medium (g/L):

52.4 g of MRS broth, 0.01 g of hemin, 0.001 g of resazurin, 10% (v/v) of clarified rumen fluid, added with distilled water to 1 L, pH 7.2±0.1, sterilized at 115° C. for 25 min, and then used after autoclaving. An additional 15 g of agar was added to obtain solid medium

2) Preparation Method of Limosilactobacillus reuteri GOLDGUT-LR99 Bacterial Powder Feed

Limosilactobacillus reuteri was activated twice in the modified MRS broth, then inoculated into the modified MRS liquid medium with the same composition at an inoculum size of 0.1%-10%, and anaerobically incubated at 37° C. for 2 days. After the bacterial solution was centrifuged at 10,000 rpm at 4° C. for 2 minutes to collect the precipitated cells, the precipitated cells were washed with a PBS buffer solution, resuspended in a solution containing 15-25% skim milk powder at a ratio of 5:1 and freeze-dried, and the number of viable bacteria was tested.

### 3) Design of Animal Experiment

18 SPF-grade wild male C57BL/6J mice were adopted, randomly divided into three groups, with six mice in each group, and adaptively fed for one week;

On experiment days 1-5, each mouse of GOLDGUT-LR99 group was given mixed quantitative bacterial powder feed (viable count 2×10⁹ CFU/day/PCS); mice in the control group and model group were given a sterile powder feed daily.

On days 6-15 after the start of the experiment, all mice except the control group were stimulated with water avoidance stress (WAS) and the mice in the control group were stimulated with pseudo water avoidance stress.

Endpoint sampling: a. colon.

### 4) Indicator Detection

### a) Behavioral Detection: Elevated Plus Maze Testing;

### b) Colorectal Distention Experiment

After 12 hours of fasting but not stopping water on day 20 of the experiment, the visceral sensitivity of the mice was determined using abdominal wall reflex (AWR) induced by colorectal dilation (CRD);
after intraperitoneal injection of urethane, the mice were anesthetized, a urinary catheter coated with paraffin oil was inserted into the colon and rectum of the mice; the tail end of the sacculus was 1 cm away from the anal orifice; the catheter was fixed on the tail of the mice with tape, and the mice were allowed to adapt for 20 minutes after they were awake;
the tail end of the urinary catheter was connected to a syringe through a three-way tube, and the colon and rectum were dilated using gas pressure values of 0.05, 0.1, 0.15, and 0.2 mL respectively; the target pressure was reached within 3 seconds and maintained for 20 seconds, with a stimulation interval of 4 minutes; under the condition of unknown pressure, the withdrawal of abdominal wall reflex in mice was observed and scores were recorded; each pressure was repeated three times. The AWR score was used to evaluate the visceral sensitivity of mice.

Scoring criteria: score of 0: there was no obvious reaction to dilation; score of 1: except for the shaking of the mouse head at the beginning of stimulation, there was no obvious contraction of the mouse abdominal wall during rectal stimulation; score of 1: slight abdominal muscle contraction occurred in the mouse during stimulation, but the abdomen was not lifted; score of 3: the abdominal muscle contraction was strong in the mouse during stimulation, and the abdomen left the platform and was lifted; score of 4: the abdominal muscle exhibited the strongest contractions in the mouse during stimulation, the body was arched and the pelvis left the platform and was lifted.

### 3.2.2 Experimental Results:

1) Improvement on Weight Gain Rate of IBS Mice by Limosilactobacillus reuteri GOLDGUT-LR99:

The results in Table 11 show that Limosilactobacillus reuteri GOLDGUT-LR99 could significantly increase the weight gain rate of mice, and the weight gain of mice in the model group was significantly inhibited.

TABLE 11
Net weight gain rate of mice in each group (%)

| Mouse ID | No. 1 | No. 2 | No. 3 | No. 4 | No. 5 | Average | P-value | |
|---|---|---|---|---|---|---|---|---|
| Control group | 8.61 | 6.31 | 5.18 | 6.7 | 10.01 | 7.36 ± 1.57 | <0.0001 | |
| Model group | −1.8 | 0.39 | 0.6 | 0.26 | −0.9 | −0.29 ± 0.84 | | <0.0001 |
| GOLDGUT-LR99 | 8.66 | 14.64 | 6.49 | 7.62 | 13.7 | 10.22 ± 3.02 | | |
| group | | | | | | | | |

2) Improvement on Visceral Hypersensitivity of Mice by Limosilactobacillus reuteri GOLDGUT-LR99

The data in Table 12 shows that Limosilactobacillus reuteri GOLDGUT-LR99 could obviously reduce the pain response of mice in the model group and obviously improve visceral hypersensitivity symptoms.

TABLE 12
Pain evaluation score of model mice

| Group | ID | 0.05 mL | 0.1 mL | 0.15 mL | 0.2 mL |
|---|---|---|---|---|---|
| Control group | No. 1 | 0 | 1.5 | 2.5 | 4 |
| | No. 2 | 0 | 0 | 1.5 | 3 |
| | No. 3 | 1 | 1 | 2 | 4 |
| | No. 4 | 0.5 | 1 | 2.5 | 4 |
| | No. 5 | 0 | 0 | 2 | 3.5 |
| Model group | No. 1 | 0 | 2 | 3 | 4 |
| | No. 2 | 1 | 2.5 | 3.5 | 4 |
| | No. 3 | 1 | 2.5 | 3 | 4 |
| | No. 4 | 1 | 2.5 | 3.5 | 4 |
| | No. 5 | 0 | 2 | 3 | 4 |
| GOLDGUT- | No. 1 | 0 | 1 | 2 | 3 |
| LR99 Group | No. 2 | 0.5 | 1 | 1.5 | 3.5 |
| | No. 3 | 0 | 0.5 | 0.5 | 2 |
| | No. 4 | 0.5 | 1.5 | 3 | 4 |
| | No. 5 | 0 | 1.5 | 2 | 4 |

3) Improvement on Anxiety States of Model Mice by Limosilactobacillus reuteri GOLDGUT-LR99

The results in Tables 13 and 14 show that Limosilactobacillus reuteri GOLDGUT-LR 99 could significantly increase the movement distance in opening arms and the number of times the model mice entered the opening arms in the elevated plus maze testing of model mice, and relieve the anxiety states of IBS mice.

TABLE 13
Movement distance (cm) in opening arms in each group after intervention

| Mouse ID | No. 1 | No. 2 | No. 3 | No. 4 | No. 5 | Average | P-value | |
|---|---|---|---|---|---|---|---|---|
| Control group | 37.93 | 37.5 | 43.75 | 31.58 | 39.39 | 38.03 ± 3.91 | <0.0001 | |
| Model group | 17.24 | 21 | 11.11 | 23.08 | 13.16 | 17.12 ± 4.53 | | 0.0002 |
| GOLDGUT-LR99 | 30.56 | 31.58 | 36.11 | 34 | 41.67 | 34.78 ± 3.95 | | |
| group | | | | | | | | |

TABLE 14
Number of times each group of mice entered opening arms after intervention (%)

| Mouse ID | No. 1 | No. 2 | No. 3 | No. 4 | No. 5 | Average | P-value | |
|---|---|---|---|---|---|---|---|---|
| Control group | 6.83 | 7.07 | 7.91 | 7.15 | 11.14 | 8.02 ± 1.60 | 0.04 | |
| Model group | 0.57 | 2.85 | 0.84 | 1.48 | 5.34 | 2.22 ± 1.75 | | 0.004 |
| GOLDGUT-LR99 | 6.77 | 12.62 | 19.1 | 9.25 | 6.62 | 10.87 ± 4.65 | | |
| group | | | | | | | | |

4) Significant Increase in the Expression of Tight Junction Protein Related Genes in Colon by Limosilactobacillus reuteri GOLDGUT-LR99

The results in Table 15 show that the intervention of Limosilactobacillus reuteri GOLDGUT-LR99 could significantly increase the expression level of tight junction protein related gene ZO-1 in the colon of IBS mice, significantly enhance the integrity of the intestinal wall of IBS mice, and significantly improve the barrier function of the intestinal wall.

TABLE 15
Expression level of gene ZO-1 in mice colon after intervention of the strain

| Mouse ID | No. 1 | No. 2 | No. 3 | No. 4 | No. 5 | Average | P-value | |
|---|---|---|---|---|---|---|---|---|
| Control group | 1.63 | 2.26 | 1.53 | 1.54 | 1.61 | 1.71 ± 0.28 | 0.002 | |
| Model group | 1.09 | 1.24 | 0.92 | 1.23 | 0.91 | 1.08 ± 0.14 | | 0.03 |
| GOLDGUT-LR99 | 1.87 | 1.48 | 1.35 | 1.39 | 1.46 | 1.51 ± 0.19 | | |
| group | | | | | | | | |

What is claimed is:

1. Limosilactobacillus reuteri GOLDGUT-LR99 with a deposit number of CGMCC No. 29346.

2. A microbial agent, wherein an active ingredient of the microbial agent is the Limosilactobacillus reuteri GOLDGUT-LR99 of claim 1.

3. A composition, comprising the Limosilactobacillus reuteri GOLDGUT-LR99 of claim 1.

4. An application of the Limosilactobacillus reuteri GOLDGUT-LR99 of claim 1 in preparing health food or medicine for treating or improving chronic fatigue syndrome.

5. The application of claim 4, wherein the chronic fatigue syndrome comprises a symptom of inhibited body weight gain, a symptom of impaired spatial memory, and a symptom of impaired exercise property.

6. An application of the Limosilactobacillus reuteri GOLDGUT-LR99 of claim 1 in preparing health food or medicine for treating or improving irritable bowel syndrome.

7. The application of claim 6, wherein the irritable bowel syndrome comprises a symptom of inhibited body weight gain, a symptom of visceral hypersensitivity, a symptom of anxiety, and a symptom of intestinal barrier dysfunction.

8. An application of the Limosilactobacillus reuteri GOLDGUT-LR99 of claim 1 for preparing a bacterial agent for inhibiting Escherichia coli or Pseudomonas aeruginosa.
